(19) United States
(12) Patent Application Publication
(10) Pub. No.: US 2013/0183710 A1
(43) Pub. Date: Jul. 18, 2013

(54) Reference sample for quality control in molecular diagnosis

(71) Applicants: Elke Reifenberger, Sankt Augustin (DE); Petra Walter, Duisburg (DE); Christoph Petry, Neuss (DE)

(72) Inventors: Elke Reifenberger, Sankt Augustin (DE); Petra Walter, Duisburg (DE); Christoph Petry, Neuss (DE)

(73) Assignee: Siemens Healthcare Diagnostics Inc., Tarrytown, NY (US)

(21) Appl. No.: 13/817,208

(22) PCT Filed: Jul. 18, 2011
(86) PCT No.: PCT/EP2011/062204

§ 371 Date: Apr. 3, 2013

(30) Foreign Application Priority Data
Aug. 17, 2010 (EP) 10008561.2

(51) Int. Cl. C12Q 1/68

(52) U.S. Cl. CPC C12Q 1/68
USPC 435/29

(57) ABSTRACT

The present invention relates to a reference sample for quality control purposes in molecular diagnosis of biological samples, which reference sample comprises a mixture of cells from at least one reference cell line which exhibits a particular gene expression profile, and at least one type of lymphocytes. Furthermore, the invention relates to productions methods for such references samples as well as to methods of their use.

### FIELD OF THE INVENTION

The present invention relates to the quality control of kPCR- or microarray-based quantification of nucleic acids (e.g. mRNA transcripts) in tissue samples. These samples are in most cases formalin fixed and paraffin embedded samples.

### BACKGROUND OF THE INVENTION

The quality control of kPCR- or microarray-based quantification of nucleic acids (e.g. mRNA transcript based expression profiling) requires control materials that can be manufactured easily and reproducibly to assure that the entire analytical procedure, including the preanalytical procedures meet the particular requirements.

These procedures normally include a reverse transcriptase (RT) reaction, a kinetic PCR (kPCR) step and the subsequent isolation of mRNA. Depending on the readout platform the process may include other enzymatic steps, e.g. labelling of nucleic acids in case of microarray applications.

Ideally, naturally occurring tissue, particularly tumor tissue, fixed and cut into slices, is being used as control material for these types of assays. Still, the inherent variation of naturally occurring tissue precludes its use as controls in quantitative diagnostic assays. Different tumor sections may contain vastly different cell populations that make it impossible to manufacture controls at larger batch sizes with uniform compositions.

In contrast thereto, artificial samples derived from cell lines can be manufactured more uniformly, still, they do not meet the requirements of real-life assays: tumor cell lines do not necessarily contain all mRNA transcripts that are found in native tumor samples and that need to be analyzed in a diagnostic assay.

Currently, spike-in controls of purified nucleic acids are commonly used as controls in RT-kPCR or microarray applications. However, the more parameters need to be controlled in multiplex assays, the more challenging it is to provide a control for the entire set of tests. Also, these controls do not monitor the performance of the sample extraction step.

Another approach is to use paraffin embedded cells from particular cell lines. However, formalin fixed paraffin embedded (“FFPE”) samples of naturally occurring tissue samples typically contain not just one cell type, but a number of different cell types, including, e.g. tumor cells, stromal cells, endothelial cells, and normal epithelial cells.

Cell lines embedded in paraffin are commercially available mainly for us as a control for immunohistochemistry (IHC) applications; e.g. the “MAX Array Breast cancer control cell block” (Zymed, Invitrogen). This product consists of formalin fixed cell line cells embedded in paraffin. Different cell lines are deposited as individual cylindrical cores in the paraffin block. This enables a side by side comparison of different cell lines, which might show different expression of various hormone receptors for instance. For control applications, the paraffin block can be cut as any regular paraffin block on a standard microtome. The major purpose of this type of control is a differentiation between high and low staining intensities in IHC.

From US 6406840 a similar approach is known, in which a cell array comprising a plurality of tube segments obtained by cross-sectioning a tube array containing frozen viable cells is used. Each tube segment of the cell array has at least one lumen and a population of frozen viable cells of a specific type that is contained and immobilized within said lumen. The array is meant to be used for performing comparative cell-based analyses with minimum inconsistencies.

From US 2008/0090243, a “pseudo-tissue sample” is known which is a composition comprising an embedding medium, e.g. paraffin, and a clonal population of cells or multiple clonal populations of cells. All of the cells of a clonal population of cells within a pseudo-tissue sample may be fixed cells, e.g. with formaldehyde, and may have been collected into an aggregation in order to form such pseudo tissue sample.

US 2008/0090243 further describes that histological specimens can be obtained from said pseudo-tissue samples. These histological specimens can be investigated microscopically or other histologically, for example in order to compare a biological sample with said reference histological specimen. By identifying subcellular features in the biological sample that are also present in the reference specimen, the researcher is able to correlate the phenotype of the biological sample with the genotype of the reference specimen.

It has however turned out that the approaches set forth above do not meet the requirements which are to be met in order to have a reference sample which allows a satisfying quality control of molecular diagnosis approaches of biological samples, particularly of tumor samples which undergo kPCR- or microarray-based quantification of nucleic acids.

### DEFINITIONS

The term “an object's gene expression profile which reflects the gene expression profile of a given biological sample”, as used herein, shall mean that at least some significant features of said object's gene expression profile are similar to those of the gene expression profile of a given biological sample, in such way that correlations between said object and said sample can be drawn.

The term “molecular diagnosis”, as used herein, shall refer to use of nucleic acid detection technologies for the investigation of biological samples, in order to provide data which allow a statement with respect to diagnosis, prognosis and/or prediction.

The term “transformed and/or immortalized cell line”, as used herein, refers to a permanent cell line wherein cells have been transformed or immortalized artificially, e.g. by treatment with radiation, certain chemicals, or certain viruses (like EBVm Adenovirus and so forth). In the context of the present invention, the said definition applies both for reference cell lines and lymphocyte cell lines.

The term “cancerous cell line”, as used herein, refers to a cell line which has been obtained from cancer cells, e.g. from carcinoma cells, sarcoma cells, lymphoma cells or leukemia cells. In the context of the present invention, the said definition applies both for reference cell lines and lymphocyte cell lines.

The term “biological sample”, as used herein, refers to a sample obtained from a patient. The sample may be of any biological tissue. Such samples include, but are not limited to, blood cells (e.g. lymphocytes), tissue, or core or fine needle biopsy samples. Biological samples may also include sections of tissues such as frozen or fixed sections taken for histological purposes or microdissected cells or extracellular parts thereof. A biological sample to be analyzed is tissue material from neoplastic lesion taken by aspiration or punctuation, excision or by any other surgical method leading to biopsy or resected cellular material. Such biological sample may comprise cells obtained from a patient. The cells may be found in a cell “smear” collected, for example, by a nipple aspiration, ductal lavarge, fine needle biopsy or from provoked or spontaneous nipple discharge.

As used herein, the term “reference cell line” relates to a cell line which can be used in the reference sample according to the invention as it reflects at least some of the properties of the biological sample which is to be investigated.

The term “lymphocytes” as used herein, shall refer to white blood cells from the vertebrate immune system. They can be divided into large granular lymphocytes, which include natural killer cells, and small lymphocytes, which consist of T cells and B cells.

The term “coagulating agent”, as used herein, shall refer to an agent which brings together cells in suspension and causes the aggregation of the latter, optionally leading to a precipitation of the cells. In a preferred embodiment, said coagulating agent is thrombin.

The term “kinetic PCR” (“kPCR”), which is also referred to as “real-time PCR” or quantitative PCR (“qPCR”) refers to the quantitative detection of PCR products. Preferably, this is achieved by means of a fluorescent signal generated by the coupling of a fluorescent reporter dye (also referred to herein as a “fluorophore” or “fluorescent dye”) and a quencher moiety to the same or different oligonucleotide substrates.

The term “RT-PCR” (often erroneously mixed up with the term “real time PCR”) is a variant of the polymerase chain reaction (PCR), in which an RNA strand, e.g. from an mRNA, is first reversely transcribed into its DNA complement (complementary DNA, or cDNA) using the enzyme reverse transcriptase, and the resulting cDNA is amplified and/or detected using traditional or real-time PCR.

Examples of probes commonly used in kPCR and kRT-PCR include the following probes: Taqman probes, Molecular Beacons probes, Scorpions probes, and SYBR Green probes. Briefly, Taqman probes, Molecular Beacons, and Scorpion probes each have a fluorophore attached to the 5′ end of the probes and a quencher moiety coupled to the 3′ end of the probes. In the unhybridized state, the proximity of the fluorophore and the quencher moiety prevents the detection of fluorescent signal from the probe; during PCR, when the polymerase replicates a template on which a probe is bound, the 5′-nuclease activity of the polymerase cleaves the probe, thus, increasing fluorescence with each replication cycle. SYBR Green probes bind double-stranded DNA and upon excitation emit light; thus, as PCR products accumulate, fluorescence increases.

The term “nucleic acid microarray” (also termed DNA microarray or DNA chip) relates to a multiplex technology used in molecular biology. It consists of an arrayed series of thousands of microscopic spots of DNA oligonucleotides, called features, each containing picomoles of a specific DNA sequence, known as probes. This can be a short section of a gene or other DNA element that is used to hybridize a cDNA or cRNA sample (called target) under high-stringency conditions. Probe-target hybridization is usually detected and quantified by detection of fluorophore-, silver-, or chemiluminescence-labeled targets to determine relative abundance of nucleic acid sequences in the target. Since an array can contain tens of thousands of probes, a microarray experiment can accomplish many genetic tests in parallel. Therefore arrays have dramatically accelerated many types of investigation.

The term “Planar Waveguide” (PWG) relates to a technology which combines highly selective fluorescence detection of, e.g. fluorophore-labeled nucleic acid probes immobilized on a chip-like surface with high sensitivity. PWGs are 150 to 300 nm thin films (“chips”) made of a material with a high refractive index, such as titanium dioxide (TiO₂) or tantalum pentoxide (Ta₂O₅), that are deposited on a transparent support with a low refractive index, such as glass, silicon dioxide, or a polymer. A parallel laser light beam is coupled into the waveguiding film by a diffractive grating that is etched or embossed into the substrate. When the light propagates within the film, a strong evanescent field that is perpendicular to the direction of propagation is produced, which enters into the adjacent medium. The intensity of the evanescent field can be enhanced by increasing the refractive index of the waveguiding layer and decreasing the layer thickness. Compared to confocal excitation of the field intensity close to the surface can be increased by a factor or up to 100. The field strength decays exponentially with the distance from the waveguide surface and its penetration depth is limited to about 400 nm. This effect can be used to selectively excite only fluorophores located at or near the surface of the waveguide, which in turn results in a significant decrease in background interference that results from fluorescence emission of the solution in the well. In nucleic acid microarray applications the need for an amplification step or an additional washing step is thus avoided.

As used herein, the term “gene expression profile” refers to the relative expression level of at least one mRNA transcript or protein from at least one cell from at least one reference cell line and/or from at least one lymphocyte, optionally from at least one lymphocyte cell line. Preferably, such profile comprises the relative expression level of two or more mRNA transcripts or proteins. A gene expression profile of a tumor sample, or a cell line, reflects the amount of each mRNA transcript and/or protein in the sample. In case all mRNA transcripts form part of the gene expression profile (and probably rRNA, tRNA, and non-coding RNA, too), the gene expression profile reflects the transcriptome.

As used herein, the term “joint gene expression profile” refers to the relative expression level of at least one mRNA transcript or protein from two or more cells from two or more reference cell lines and/or lymphocytes, the latter optionally from at least one lymphocyte cell line.

As used herein, the term “quantitatively defined mixture” means that the mixture of cells from the at least two cell lines is thoroughly controlled. A good definition of the quantitative composition of cells in the reference sample is helpful to ensure that the ratio of the gene expression profiles in the reference sample corresponds to the ratio of gene expression profiles in a certain tissue type, e.g. a tumor sample which is to be investigated.

The term “tumor” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. The term “cancer” as used herein includes carcinomas, (e.g. carcinoma in situ, invasive carcinoma, metastatic carcinoma) and pre-malignant conditions, neomorphic changes independent of their histological origin. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included. Examples of cancers include, but are not limited to colorectal cancer, lung cancer, ovarian cancer, cervical cancer, stomach cancer, pancreatic cancer, head and neck cancer and/or breast cancer.

The term “prediction”, as used herein, relates to an individual assessment of the malignancy of a tumor, or to the expected survival rate (DFS, disease free survival) of a patient, if the tumor is treated with a given therapy, e.g. surgery, chemotherapy, radiotherapy and/or targeted therapy (i.e., drug-based therapy which binds to specific tumor targets, e.g. growth factors, receptors or the like).

In contrast thereto, the term “prognosis” relates to an individual assessment of the malignancy of a tumor, or to the expected survival rate (DFS, disease free survival) of a patient, if the tumor remains untreated.

### OBJECT OF THE INVENTION

It is one object of the present invention to provide a reference sample which improves the quality control of molecular diagnosis approaches of biological samples, particularly of tumor samples which undergo kPCR- or microarray-based quantification of nucleic acids.

It is another object of the present invention to provide a method for preparing a reference sample for quality control purposes in molecular diagnosis.

It is yet another object of the present invention to provide a method for molecular diagnosis which improves the quality control of molecular diagnosis approaches of biological samples.

These objects are met with methods and means according to the independent claims of the present invention. The dependent claims are related to preferred embodiments.

### SUMMARY OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” an and the include singular and/or plural referents unless the context clearly dictates otherwise. It is moreover to be understood that, in case parameter ranges are given which are delimited by numeric values, the ranges are deemed to include these limitation values.

According to the invention, a reference sample for quality control purposes in molecular diagnosis of biological samples is provided, which reference sample comprises a mixture of cells from
a) at least one reference cell line which exhibits a particular gene expression profile, and
b) at least one type of lymphocytes.

The inventors of the present invention have surprisingly found that, when adding lymphocytes to a reference sample which contains cells from at least one reference cell line which exhibits a particular gene expression profile, the said reference probe achieves a much better performance, i.e., a better match with the gene expression profile of the biological sample under examination is obtained.

The inventors realized that many biological samples contain transcripts, e.g. mRNA, from immune cells, like lymphocytes, although these cells do not originally occur there. This is probably due to the fact that on many occasions the immune system sends immune cells into a given tissue in order to evoke an immune response. In case a sample is taken from such tissue and investigated with respect to the gene expression profile, the said immune cells leave their molecular fingerprint, as these cells produce mRNAs, like the tissue cells of the sample which is investigated.

The above applies particularly in cases where an immune response is currently taking place in the tissue that is investigated, for example because of an inflammatory process, an infection, an autoimmune process, or a malignant process, e.g. a tumor.

Particularly in the latter the immune system sends lymphocytes into the tumor, where these cells are involved in tumor cell-killing tasks, like ADCC (Antibody Dependent Cytotoxicity), CDC (Complement Dependent Cytotoxicity) Antibody-Dependent Apoptosis or Antibody-Dependent Opsonisation.

This means that tumor samples are particularly contaminated with mRNA from immune cells. For this reason, the present invention is particularly useful in molecular cancer diagnosis.

Examples for such reference cell lines are shown in Table 1.

TABLE 1

| Name | Cell type |
|---|---|
| MFC7 (ATCC HTB-22) | Epithelial breast cancer cell line |
| BT474 (ATCC HTB-20) | Epithelial breast cancer cell line |
| T47D (ATCC HTB-133) | Epithelial breast cancer cell line |

Preferably, said reference sample has the shape of a block, similar to those which are routinely used for the analysis of biological samples in immunohistochemistry approaches or histopathology.

In a preferred embodiment of the present invention, the molecular diagnosis approach is at least one selected from the group consisting of:
kinetic PCR (kPCR), preferably kinetic RT-PCR (kRT-PCR);
nucleic acid microarray analysis, preferably with a Planar Waveguide Microarray (PWG); and/or
in situ hybridization.

In another preferred embodiment of the present invention, the mixture of cells is fixed with a fixing agent, said fixing agent preferably comprising formaldehyde. Equally preferred is that the mixture of cells is embedded in an embedding agent, said embedding agent preferably comprising paraffin.

In clinical pathology, patient samples are usually formaldehyde fixed and paraffin embedded shortly after excision. Standard pathology protocols are thus adapted to FFPE-treated samples, and the majority of tissue and tumor samples stored in sample collections and databases are FFPE-treated samples.

It is thus beneficial if the reference sample according to the invention is fixed with a fixing agent, like formaldehyde, and/or embedded in an embedding agent, like paraffin, mainly in order to contribute to a realistic behavior of the reference sample, e.g. under the microtome, so that slices can be produced which have a similar look and feel as real tissue or tumor slices.

In another preferred embodiment of the present invention, the mixture of cells used for the reference sample is a quantitatively defined mixture.

The major advantage of this approach is that by quantitatively defining the mixture of cells which is comprised in the reference probe the joint gene expression profile of the reference sample can be finetuned in order to faithfully reflect the gene expression profile of a given biological sample, e.g. a tumor sample.

This applies preferably to the fraction of cells from the reference cell lines, not necessarily to the lymphocyte cell fraction, at least not in case the latter has been obtained from a blood sample, for the reasons set forth below.

In yet another preferred embodiment of the present invention, the lymphocytes are obtained from at least one source selected from the group consisting of
blood sample;
primary lymphocyte cell line;
transformed and/or immortalized lymphocyte cell line; and/or
cancerous lymphocyte cell line.

Such blood sample can preferably be obtained from a blood donor, e.g. from the buffy coat phase of a blood sample provided by the latter. A buffy coat is the thin fraction of a blood sample which, after density gradient centrifugation, forms between the serum phase and the red blood cell phase. It consists merely of lymphocytes and platelets and forms about one percent of the blood sample.

However, a blood sample, preferably a buffy coat sample, comprises an undefined mixture of different lymphocyte cell types in varying concentrations. This means that when lymphocytes from a blood sample, are being used, it is difficult to add the lymphocytes to the mixture of cells in a quantitatively defined fashion. Thus, in most cases only the cells obtained from the reference cell lines can be provided in a quantitatively defined fashion in this embodiment.

Alternatively, the said lymphocytes can be obtained from cell culture, e.g. from a primary lymphocyte cell culture or from a transformed lymphocyte cell line.

This alternative allows the preparation of a defined cell culture with only one cell type in a given concentration. For this reason, all cell types (i.e. both the cells obtained from the reference cell lines and the lymphocytes) can be provided in a quantitatively defined fashion in this embodiment.

Furthermore, this alternative also allows to select a lymphocyte cell line according to a particular gene expression profile.

Examples for such transformed lymphocyte cell lines are shown in Table 2.

TABLE 2

| Name | Cell type | Other features |
|---|---|---|
| ARH-77 (ATCC CRL-1621) | B-cell | EBV-transformed |

In yet another preferred embodiment of the present invention, the at least one reference cell line, and/or the at least one lymphocyte cell line, respectively, cells of which are comprised in the sample, has been selected in such way that its gene expression profile reflects at least part of the gene expression profile of a given biological sample.

Preferably, at least two reference cell lines are being used in the reference sample according to the present invention, in order to better reflect the respective properties of the biological sample, which is in many cases heterogeneous, e.g. consisting of at least two different cell types plus lymphocytes. In this embodiment, the reference cell lines are being selected in such way that their joint gene expression profile reflects the gene expression profile of said biological sample.

This selection step can for example be done as follows: Cells from cell lines are pre-selected based on their origin (tumor entity, lymphatic, etc.). Subsequently, RNA from preselected cell lines is isolated and analyzed with a high density expression profiling method, e.g. with an Affymetrix GeneChip, to quantify as many transcripts as possible. Based on the results those cell lines are selected which contain the transcripts to be measured in the diagnostic test in a reasonable quantity. Selected cell lines are cultivated and harvested separately. Subsequently, the different cell lines are mixed in adequate quantities which ensure that the ratio of the transcripts in the cell line mixture corresponds to the ratio of transcripts in a certain tissue type (e.g. breast tumor samples with good prognosis, or breast tumor samples with bad prognosis).

In yet another preferred embodiment of the present invention, the biological sample is at least one tissue sample in which a process takes place, or has taken place, which is selected from the group consisting of
an inflammatory process,
an infection,
an autoimmune process, and
a malignant process, e.g. a tumor.

Preferably, said malignant process, or tumor, is at least one selected from the group consisting of breast cancer, colorectal cancer, lung cancer, ovarian cancer, cervical cancer, stomach cancer, pancreatic cancer, lymphoma, melanoma, sarcoma, head and neck cancer, and prostate cancer.

In yet another preferred embodiment of the present invention, the at least one reference cell line is selected from the group consisting of
a primary cell line,
a transformed and/or immortalized cell line, and
a cancerous cell line.

As mentioned above, the said reference cell lines are selected in such way that their properties, in particular their gene expression profile, reflects at least part of the properties of the given biological sample.

In order to prepare a reference sample for a breast cancer biopsy, for example, it is useful to use one or more breast cancer cell lines as reference cell lines.

Similar principles are applicable in case the biological sample is a colorectal cancer sample, a lung cancer sample, an ovarian cancer sample and so forth.

In case the respective cancer which forms the biological sample can be better characterized prior to molecular analysis, e.g by histopathology, it is furthermore useful to select the reference cells lines, or the respective reference sample, according to this information. If, e.g. histopathology reveals that a breast tumor sample is EGF (Epidermal Growth Factor) negative, the respective reference sample can be selected, or designed, in such way that it contains cells from breast cancer cell lines which are also EGF negative.

In yet another preferred embodiment of the present invention, the malignant process, or tumor, is at least one selected from the group consisting of:
a tumor with a good prediction towards a given therapy,
a tumor with a bad prediction towards a given therapy.

In yet another preferred embodiment of the present invention, the reference sample furthermore comprises at least one type of extracellular matrix protein.

Such extracellular matrix proteins are for example collagens, elastins, fibronectins, laminins and/or proteoglycans. They contribute to a more realistic behavior of the reference sample, e.g. under the microtome, so that slices can be produced which have a similar look and feel as real tissue or tumor slices, or upon lysis of the sample in order to prepare the molecular diagnosis.

In another embodiment of the present invention a specimen for histological and/or molecular biological analysis is provided, said specimen being obtainable from a reference sample according the invention by means of
microslicing, e.g. with a microtome,
microdissection, e.g. with a laser microdissection apparatus, and/or
needle aspiration.

In still another embodiment of the present invention, a method for preparing a reference sample for quality control purposes in molecular diagnosis is provided, which method comprises at least the following steps:
cultivating cells from at least one reference cell line;
mixing the cultivated cells with at least one type of lymphocytes; and
preparing a sample from the mixed cells.

In a preferred embodiment of this method, and prior to the cultivation step, the at least one reference cell line, and/or at least one lymphocyte cell line, respectively, cells of which are comprised in the reference sample, is selected in such way that its gene expression profile reflects at least part of the gene expression profile of a given biological sample.

In yet another preferred embodiment of this method the gene expression profile of cells of the at least one reference cell line and/or at least one lymphocyte cell line is determined prior to the selection step.

In yet another preferred embodiment of this method, the cells are mixed in a quantitative manner. The major advantage of this approach is that by quantitatively defining the mixture of cells which is comprised in the reference probe the joint gene expression profile of the reference sample can be finetuned in order to faithfully reflect the gene expression profile of a given biological sample, e.g. a tumor sample.

This applies preferably to the fraction of cells from the reference cell lines (i.e. when two or more reference cell lines are used), not necessarily to the lymphocyte cell fraction, at least not in case the latter has been obtained from a blood sample. However, if the lymphocytes are obtained from lymphocyte cell lines, they can also be added to the mixture in a quantitative manner.

In still another preferred embodiment of this method, the sample is prepared by at least one step selected from the group consisting of:
binding the cells to one another, preferably with a coagulating agent,
fixing the cells with a fixing agent, said fixing agent preferably comprising formaldehyde, and
embedding the cells in an embedding agent, said embedding agent preferably comprising paraffin.

In another embodiment of the present invention, a kit for quality control purposes in molecular diagnosis of biological samples is provided, said kit comprising at least two reference samples according to the invention, wherein each reference sample reflects the properties of a different biological sample.

According to this approach, said kit may, for example, contain two reference samples which represent, in their gene expression profile, two different tumor types, e.g. a high risk breast tumor and a low risk breast tumor.

Furthermore, a quality control method for molecular diagnosis of biological samples is provided, which method comprises at least the following steps:
a) obtaining a biological sample,
b) investigating said biological sample by means of molecular diagnosis, and
c) comparing the results of step b) with results obtained from investigating a reference sample according to the invention.

### BRIEF DESCRIPTION OF THE EXAMPLES AND DRAWINGS

Additional details, features, characteristics and advantages of the object of the invention are disclosed in the subclaims, and the following description of the respective figures and examples, which, in an exemplary fashion, show preferred embodiments of the present invention. However, these drawings should by no means be understood as to limit the scope of the invention.

### Example

### Preparation of a Breast Cancer Reference Sample According to the Invention

For preparing a cell line block for application in breast cancer prognosis the following cell lines were selected due to their (partially known) expression profiles:
MFC7 (ATCC HTB-22),
BT474 (ATCC HTB-20),
T47D (ATCC HTB-133), and
ARH 771 (ATCC CRL-1621).

Cell lines MCF7, BT474, and T47D are epithelial breast cancer cell lines. ARH-77 is a B-lymphoblast cell line (EBV-transformed) from peripheral blood. For cell line-block preparation each cell line is cultivated separately according to the ATCC proposals. 1×10⁷ cells are harvested and washed twice with PBS-buffer. Subsequently the cells are mixed in a ratio of 1:1:1:1. Subsequently, the mixed cell suspension is centrifuged carefully and resuspended in PBS-buffer supplemented with Ca²⁺ and Mg²⁺ ions. The cell mixture is centrifuged again and the cell pellet resuspended in 250 μl human plasma. After addition of human thrombin the cell pellet is coagulated for at least 5 min. The resulting cell block is fixed over night in 4% PBS buffered formalin. After fixation the cell block is dehydrated in ascending order of ethanol up to 100%. The cell block is then incubated in Xylol for 1 hour (until the block is transparent) and subsequently embedded in paraffin. 5 μm or 10 μm sections are prepared with a standard microtome.

### FIGURES

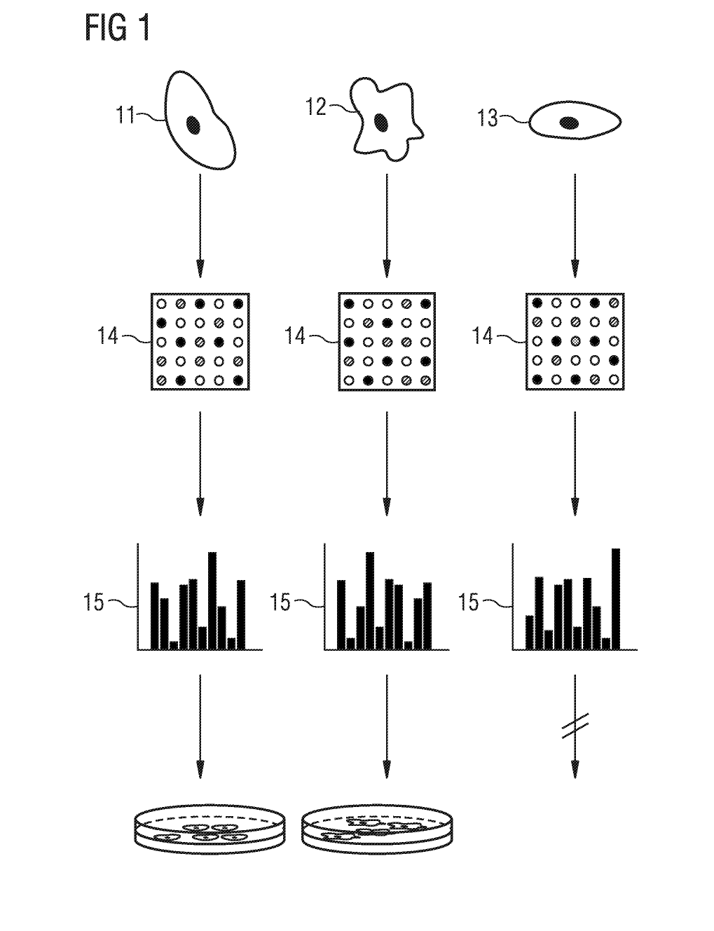

FIG. 1 shows an exemplary process of selecting reference cell lines which are to be used for preparing a reference sample according to the invention. Cells 11, 12 and 13 from reference cell lines which have been pre-selected based on their origin (tumor entity, lymphatic, etc.) are used to isolate mRNA, which is then analyzed with a GeneChip 14, to quantify as many transcripts as possible, and respective gene expression profiles 15 are produced. Based on the results reference cell lines 11 and 12 are selected, because their gene expression profiles 15 reflect at least part of the gene expression profile of the biological sample which is to be diagnosed.

It is beneficial if the joint gene expression profile of both reference cell lines reflects the gene expression profile of said biological sample.

Reference cell line 13 is discarded, because the analysis revealed that its gene expression profile does not reflect even part of the gene expression profile of said biological sample. Selected reference cell lines 11 and 12 are cultivated separately.

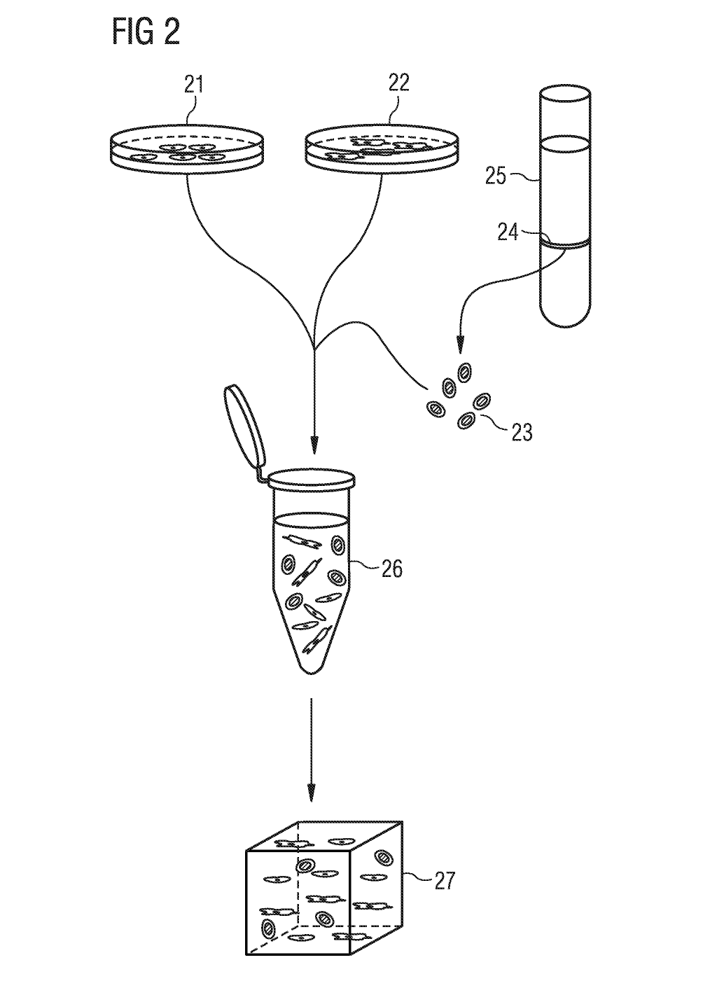

FIG. 2 shows an exemplary process of preparing a reference sample according to the invention. Cultivated reference cell lines 21 and 22 are harvested and mixed in a quantitative manner.

Lmphocytes 23 are being isolated from the buffy coat 24 of a blood sample 25 and mixed with the harvested cells from cultivated reference cell lines 21 and 22 in a reaction vessel 26.

The cell mixture is centrifuged and the cell pellet is coagulated, e.g. by addition of thrombin. The resulting cell block 27, which represents the reference sample according to the invention, is fixed and embedded in paraffin.

1. A reference sample for quality control purposes in molecular diagnosis of biological samples, which reference sample comprises a mixture of cells from
a) at least one reference cell line which exhibits a particular gene expression profile, and
b) at least one type of lymphocytes.

2. The reference sample according to claim 1, wherein the molecular diagnosis approach is at least one selected from the group consisting of:
kinetic PCR (kPCR), preferably kinetic RT-PCR (kRT-PCR),
nucleic acid microarray analysis, preferably with a Planar Waveguide Microarray (PWG), and
in situ hybridization.

3. The reference sample according to claim 1, wherein the mixture of cells is
a) fixed with a fixing agent, said fixing agent preferably comprising formaldehyde, and/or
b) embedded in an embedding agent, said embedding agent preferably comprising paraffin.

4. The reference sample according to claim 1, wherein the mixture of cells used for the reference sample is a quantitatively defined mixture.

5. The reference sample according to claim 1, wherein the lymphocytes are obtained from at least one source selected from the group consisting of:
blood sample,
primary lymphocyte cell line,
transformed and/or immortalized lymphocyte cell line, and
cancerous lymphocyte cell line.

6. The reference sample according to claim 1, wherein the at least one reference cell line and/or the at least one lymphocyte cell line, respectively, has been selected in such way that its gene expression profile reflects at least part of the gene expression profile of a given biological sample.

7. The reference sample according to claim 1, wherein the biological sample is at least one tissue sample in which a process takes place, or has taken place, which is selected from the group consisting of:
a) an inflammatory process,
b) an infection,
c) an autoimmune process, and
d) a malignant process.

8. The reference sample according to claim 1, wherein said malignant process, is at least one selected from the group consisting of:
breast cancer,
colorectal cancer,
lung cancer,
ovarian cancer,
cervical cancer,
stomach cancer,
pancreatic cancer,
lymphoma,
melanoma,
sarcoma,
head and neck cancer, and
prostate cancer.

9. The reference sample according to claim 1, wherein the at least one reference cell line is selected from the group consisting of:
a) a primary cell line,
b) a transformed and/or immortalized cell line, and
c) a cancerous cell line.

10. The reference sample according to claim 1, which sample furthermore comprises at least one type of extracellular matrix protein.

11. A specimen for histological or molecular biological analysis, said specimen being obtainable from a reference sample according to claim 1 by means of:
microslicing, e.g. with a microtome,
microdissection, e.g. with a laser microdissection apparatus, and/or
needle aspiration.

12. A method for preparing a reference sample for quality control purposes in molecular diagnosis, which method comprises at least the following steps:
cultivating cells from at least one reference cell line,
mixing the cultivated cells with at least one type of lymphocytes, and
preparing a sample from the mixed cells.

13. The method according to claim 12, wherein, prior to the cultivation step, the at least one reference cell line, and/or at least one lymphocyte cell line, is selected in such way that its gene expression profile reflects at least part of the gene expression profile of a given biological sample.

14. The method according to claim 12, wherein the gene expression profile of cells of the at least one reference cell line and/or at least one lymphocyte cell line is determined prior to the selection step.

15. The method according to claim 12, wherein the cells are mixed in a quantitative manner.

16. The method according to claim 12, wherein the sample is prepared by at least one step selected from the group consisting of
binding the cells to one another, preferably with a coagulating agent;
fixing the cells with a fixing agent, said fixing agent preferably comprising formaldehyde; and
embedding the cells in an embedding agent, said embedding agent preferably comprising paraffin.

17. A kit for quality control purposes in molecular diagnosis of biological samples, said kit comprising at least two reference samples according to claim 1, wherein each reference sample reflects the properties of a different biological sample.

18. A quality control method for molecular diagnosis of biological samples, which method comprises at least the following steps:
a) obtaining a biological sample,
b) investigating said biological sample by means of molecular diagnosis, and
c) comparing the results of step b) with results obtained from investigating a reference sample according to claim 1.
